(19) United States
(12) Patent Application Publication
(10) Pub. No.: US 2008/0139458 A1
(43) Pub. Date: Jun. 12, 2008

(54) Methods of Treatment For Injured or Diseased Joints

(76) Inventors: Gregory D. Jay, Norfolk, MA (US); Khaled A. Elsaid, Kingston, RI (US)

Correspondence Address: CLARK & ELBING LLP, 101 FEDERAL STREET, BOSTON, MA, 02110, US

(21) Appl. No.: 11/792,259

(22) PCT Filed: Dec. 1, 2005
(86) PCT No.: PCT/US05/43311

§ 371 Date: Sep. 10, 2007

Related U.S. Application Data
(60) Provisional application No. 60/633,220, filed on Dec. 3, 2004.

(51) Int. Cl. A61K 38/14; A61P 19/02

(52) U.S. Cl. 514/8

(57) ABSTRACT

The invention features pharmaceutical compositions that include lubricin, a compound that inhibits an enzyme selected from the group consisting of neutrophil elastase, cathepsin B, cathepsin K, cathepsin L, cathepsin S, papain, trypsin, chymotrypsin, subtilisin, pepsin, bromelain, ficin, Protease A, Protease B, Protease D, granzyme A, granzyme B, granzyme K, pepsin, thermolysin, pronase, dipeptidyl peptidase IV, and pancreatin, and a pharmaceutically acceptable excipient. Another aspect of the invention features methods of lubricating a joint in a mammal by contacting the joint with a pharmaceutical composition of the invention. The invention also features methods of inhibiting adhesion formation between a first surface and a second surface in a mammal (e.g., between injured tissues or between injured tissue and an artificial device).

### CROSS-REFERENCE TO RELATED APPLICATIONS

This application claims priority under 35 U.S.C. § 371 from international application PCT/US2005/043311, filed Dec. 1, 2005, which claims priority under 35 U.S.C. § 119 from U.S. Provisional Patent Application No. 60/633,220, filed Dec. 3, 2004, both of which are incorporated by reference herein.

### BACKGROUND OF THE INVENTION

The present invention relates to the lubrication of mammalian joints.

Rheumatoid arthritis (RA) and post-traumatic knee joint synovitis (KJS) are common forms of joint disease. Factors which contribute to the development of RA and KJS include previous damage to the joint through injury or surgery and the age of the joint (i.e., “wear and tear” of the articulating surfaces of the joint). Current methods of treatment are directed to relieving pain and other symptoms of RA or KJS by administering, for example, analgesics and anti-inflammatory drugs.

Also described for the treatment of injured or diseased joints are methods in which a lubricant is applied directly to the injured or arthritic joint. Lubricin, also known as proteoglycan 4 (PRG4), articular cartilage superficial zone protein (SZP), megakaryocyte stimulating factor precursor, or tribonectin (Ikegawa et al., Cytogenet. Cell. Genet. 90:291-297, 2000; Schumacher et al., Arch. Biochem. Biophys. 311:144-152, 1994; Jay and Cha, J. Rheumatol., 26:2454-2457, 1999; and Jay, WIPO Int. Pub. No. WO 00/64930) is a mucinous glycoprotein found in the synovial fluid (Swann et al., J. Biol. Chem. 256:5921-5925, 11981). Lubricin provides boundary lubrication of congruent articular surfaces under conditions of high contact pressure and near zero sliding speed (Jay et al., J. Orthop. Res. 19:677-87, 2001). These lubricating properties have also been demonstrated in vitro (Jay, Connect. Tissue Res. 28:71-88, 1992). Cells capable of synthesizing lubricin have been found in synovial tissue and within the superficial zone of articular cartilage within diarthrodial joints (Jay et al., J Rheumatol. 27:594-600, 2000).

In U.S. patent application Ser. No. 10/038,694 are described methods of promoting lubrication between two juxtaposed biological surfaces using lubricin, or fragments thereof. In U.S. Pat. No. 6,743,774 are described lubricin (tribonectin) analogs and methods for lubricating a mammalian joint. In a recent report (Englert et al., Trans. Orthop. Res. 29:189, 2003), the reduction of integration of opposing cartilage surfaces by components in synovial fluid was described and it was suggested that this reduction in integration was, at least in part, lubricin mediated.

Synovial fluids (SF) aspirated from patient populations diagnosed with KJS or RA exhibit compromised SF boundary lubricating ability, which is provided by lubricin. The SF aspirates from these patient populations show a release of articular cartilage damage markers (Elsaid et al., Osteoarthritis Cartilage 11:673-680, 2003). In addition, the elimination of the lubricating activity of molecules of the synovial fluid by trypsin has been described (Jay and Cha, J. Rheumatol., 26:2454-2457, 1999).

### SUMMARY OF THE INVENTION

Described herein is the loss of synovial fluid's boundary lubricating ability and chondroprotection in patients with RA and KJ due to the action of cathepsin B (CB) and/or neutrophil elastase (NE) S. It is proposed that inhibition of one or both of these enzymes, or other protease inhibitors that degrade the lubricating ability of lubricin, may retard the loss of SF's boundary lubricating ability and that this inhibition, alone or in combination with the application of lubricin to a mammal's articulating joint as a lubricating agent, is useful for the treatment of patients suffering from joint disease or injury.

Accordingly, in a first aspect, the invention features a method of lubricating a joint in a mammal that includes contacting the joint with lubricin and administering to the mammal a compound that inhibits an enzyme selected from the group consisting of neutrophil elastase, cathepsin B, cathepsin K, cathepsin L, cathepsin S, papain, trypsin, chymotrypsin, subtilisin, pepsin, bromelain, ficin, Protease A, Protease B, Protease D, granzyme A, granzyme B, granzyme K, pepsin, thermolysin, pronase, dipeptidyl peptidase IV, and pancreatin. Preferably, the joint is an articulating joint of a human.

The compound can be administered orally, rectally, intravenously, subcutaneously, or as an inhalant. Preferably, the compound is administered locally at the site of the injured or diseased joint, such as, for example, by direct injection into synovial fluid at the region of interest. The compound can be administered before, during, or after treatment of the joint with lubricin.

In another aspect, the invention features a method of inhibiting adhesion formation between a first surface and a second surface in a mammal that includes placing lubricin between the first and second surfaces in an amount sufficient to prevent adhesion of the surfaces and administering to the mammal a compound that inhibits an enzyme selected from the group consisting of neutrophil elastase, cathepsin B, cathepsin K, cathepsin L, cathepsin S, papain, trypsin, chymotrypsin, subtilisin, pepsin, bromelain, ficin, Protease A, Protease B, Protease D, granzyme A, granzyme B, granzyme K, pepsin, thermolysin, pronase, dipeptidyl peptidase IV, and pancreatin.

In one embodiment, the first surface and the second surface are both injured tissues. In another embodiment, the first or second surface is an artificial device, such as, for example, an orthopedic implant. In another embodiment, the first and second surfaces are tissues injured due to a surgical incision. In yet another embodiment, the first and second surfaces are tissues injured due to trauma.

In another aspect, the invention features a pharmaceutical composition comprising lubricin; a compound that inhibits an enzyme selected from the group consisting of neutrophil elastase, cathepsin B, cathepsin K, cathepsin L, cathepsin S, papain, trypsin, chymotrypsin, subtilisin, pepsin, bromelain, ficin, Protease A, Protease B, Protease D, granzyme A, granzyme B, granzyme K, pepsin, thermolysin, pronase, dipeptidyl peptidase IV, and pancreatin; and a pharmaceutically acceptable excipient.

Therapeutic formulations may be in the form of liquid solutions or suspensions. In one embodiment, the composition is in the form of a membrane, foam, gel, or fiber. Methods well known in the art for making formulations are found, for example, in Remington: The Science and Practice of pharmacy (20th ed., ed. A. R. Gennaro A R.), Lippincott Williams & Wilkins, 2000. Biocompatible, biodegradable lactide polymer, lactide/glycolide copolymer, or polyoxyethylene-polyoxypropylene copolymers may be used to control the release of formulation components.

In an embodiment of any of the aspects of the invention, the compound inhibits cathepsin B. Cathepsin B inhibitors are known to those skilled in the art and include aldehydes, alpha-ketocarbonyl compounds, halomethyl ketones, diazomethyl ketones, (acyloxy)methyl ketones, ketomethylsulfonium salts, epoxy succinyl compounds, vinyl sulfones, aminoketones, and hydrazides (see Schirmeister et al., Chem. Rev. 97:133-171, 1997). Specific inhibitors include E-64, Z-Leu-Leu-Leu-fluoromethyl ketone (Z-LLL-FMK), Z-Phe-Phe-fluoromethyl ketone, calpain inhibitor I, calpain inhibitor II, antipain, biotin-Phe-Ala-fluoromethyl ketone, cystatin, CA-074, CA-074 methyl ester, chymostatin, leupeptin, N-methoxysuccinyl-Phe-homoPhe-fluoromethyl ketone, or a procathepsin B fragment.

In another embodiment of any of the aspects of the invention, the compound inhibits neutrophil elastase. Examples of elastase inhibitors include phenylmethanesulfonyl fluoride (PMSF), ICI 200,355, secretory leukoproteinase inhibitor, MeOSuc-Ala-Ala-Pro-Ala-CMK, Boc-Ala-Ala-Ala-NHO-Bz, and MeOSuc-Ala-Ala-Pro-Val-CMK.

### BRIEF DESCRIPTION OF THE DRAWINGS

FIG. 1 is a photograph of the friction apparatus used in some of the experiments described herein

FIG. 2 is a schematic diagram of a modified Stenton pendulum

FIG. 3a is a Western blot analysis of purified human lubricin following treatment with 0.5 U/mL of cathepsin B (CB) and probed with polyclonal anti-lubricin IgG (J108) and peanut agglutinin (PNA) linked to peroxidase. Lubricin (5 μg per well) was treated with CB to a final concentration of 0.5 U/mL, reconstitutued in 0.25 M Na acetate buffer, pH 5.5 at 37° C., and sampled after 2, 4, 6, 12, and 24 hours of treatment. The enzymatic reaction was stopped by adding E-64 to a final concentration of 100 μM. Blots were probed with PNA-peroxidase and pAb J108.

FIG. 3b is a Western blot analysis of purified human lubricin treated with 0.5 U/mL neutrophil elastase (NE). Lubricin (5 μg per well) was treated with NE to a final concentration of 0.5 U/mL reconstituted in 10 mM Tris-HCl, 100 mM CaCl₂, pH 8.8 at 37° C., and sampled after 2, 4, 6, 12, and 24 hours of treatment. The enzymatic reaction was stopped by adding PMSF to a final concentration of 1 mM. Blots were probed with PNA-peroxidase and pAb J108.

FIG. 4 is a graph showing changes in coefficient of function (Δμ±S.D.) of pooled knee joint synovitis (KJS) synovial fluid (SF) aspirates, rheumatoid arthritis (RA) SF aspirates, osteoarthritis (OA) SF aspirates supplemented with purified human lubricin and normal SF aspirates following treatment at 37° C. for 24, 48, and 96 hours. The Δμ values were an average of two experiments, each with four distinct measurements of Δμ. A “*” symbol indicates that the Δμ lubricin-supplemented, pooled KJS SF aspirates were significantly higher than the Δμ of normal SF aspirates following 24, 48, and 96 hour treatments at 37° C. (P<0.001). A “**” symbol indicates that the Δμ lubricin-supplemented, pooled RA SF aspirates were significantly higher than the Δμ of normal SF aspirates following 24, 48, and 96 hour treatments at 37° C. (P<0.001). A “***” symbol indicates that the Δμ lubricin-supplemented, pooled OA SF aspirates were significantly higher than the Δμ of normal SF aspirates following 96 hour treatments at 37° C. (P<0.001).

FIG. 5 is a graph showing cathepsin B activity in knee joint synovitis (KJS) synovial fluid (SF), rheumatoid arthritis (RA) SF, and osteoarthritis (OA) SF aspirates. A “*” symbol indicates that the cathepsin B activity in KJS SF was significantly higher than the cathepsin B activity in OA SF (P<0.005). A “**” symbol indicates that the cathepsin B activity in RA SF was significantly higher than the cathepsin B activity in OA SF (P<0.001) and KJS SF (P<0.005).

### DETAILED DESCRIPTION

The proteolytic degradation of lubricin in a mammal's articular joint increases friction between cartilage surfaces. This may lead to damage and wear, thereby predisposing the joint to the development of a degenerative disease. Secondary osteoarthritis (OA) has been shown to be predisposed by joint trauma (Gelber et al. Ann. Intern. Med. 133:321-328, 2000), obesity, or strenuous occupations. Inhibition of the cascade of events leading to the subsequent development of OA may lie in the initial loss of boundary lubrication observed following knee injury (Jay et al., J. Rheumatol. 31:557-564, 2004). Lubricin appears susceptible to proteolytic degradation by enzymes that are secreted extracellularly during the initial inflammatory phase, leading to a loss of SF's boundary lubrication evidenced in KJS SF aspirates (Jay et al., J. Rheumatol. 31:557-564, 2004).

The loss of SF boundary lubrication is also evident in RA. KJS and RA represent opposite ends of an inflammation continuum in the synovium (Pando et al., J. Rheumatol. 27:1848-1854, 2000). Infiltration of polymorphonuclear (PMN) cells is common to both clinical conditions. It is therefore plausible that proteases secreted from synoviocytes and infiltrating PMN cells are responsible for the early loss of SF's boundary lubricating ability observed in KJS, as well as the loss observed in RA. Described herein are experiments showing that CB and NE can proteolytically degrade lubricin in a time-dependent manner

### Methods

### Protease Treatment of Purified Human Lubricin and Whole BSF

Human lubricin was purified from pooled SF aspirates of patients undergoing total knee replacement as described previously (Jay et al., Gluconj. J. 18:807-815, 2001). BSF was aspirated percutaneously from the lateral aspect of radiocarpal joints of freshly slaughtered cattle with sterile 18 gauge needles after cleansing the skin with alcohol swabs. The cattle were 1 year old and of both sexes (PelFreeze Corp., Little Rock, Ark.). The BSF was centrifuged at 20,000 g at 4° C. to remove cell debris and the BSF was stored at −20° C. Aliquots of purified human lubricin (1 mL, 250 μg/1 mL), and BSF (1 mL) were subjected to protease treatments with CB, NE, α-chymotrypsin, or trypsin. These enzymes were utilized as follows: 1) 0.5 U/mL of CB (Sigma-Aldrich, Saint Louis, Mo., one unit liberates 1 nanomole of 7-amino-4-methylcoumarin from Z-Arg-Arg 7-amido-4-methylcoumarin per min at pH 6.0 at 40° C.) reconstituted in 0.25 M Na acetate buffer, pH 5.5; 2) 0.5 U/mL NE (Sigma-Aldrich, one unit is defined as the release of 1 mole of p-nitrophenol per sec at 37° C.) reconstituted in 100 mM Tris-HCl, 100 mM CaCl₂, pH 8.8; 3) 0.5 U/mL of TPCK-treated trypsin (Sigma-Aldrich, one unit causes a change in A₂₅₃ of 0.001 per min at pH 7.6 at 25° C. using BAEE as a substrate) reconstituted in 50 mM Tris HCl, 20 mM CaCl₂, pH 8.0; and 4) 0.5 U/mL of α-chymotrypsin (Sigma-Aldrich, one unit hydrolyzes 1.0 μmole of BTEE per min at pH 7.8 at 25° C.) reconstituted in 80 mM Tris-HCl, 100 mM CaCl₂, pH 7.8.

Aliquots of digested human lubricin and BSF (200 μL) were removed after 2, 4, 6, 12, and 24 hours and the reaction was stopped by adding E-64 (L-trans-epoxysuccinyl-leucyl-amido-(4-guanidino)-butane, Sigma-Aldrich) to a final concentration of 100 μM (CB) or phenylmethanesulfonyl fluoride (PMSF, Sigma-Aldrich) to a final concentration of 1 mM (NE).

Electrophoresis was performed on pre-cast SDS-PAGE 4-15% gels (BioRad, Hercules, Calif.) under reducing conditions. High molecular weight standards (BRL, Gaithersburg, Md.) were electrophoresed simultaneously with the treated human lubricin or BSF. Electrophoresis was performed at 150 V for 90 min until the wave front exited from the bottom of the gel. Western transfer to nitrocellulose was carried out under semi-dry conditions at 20 V for 40 min. The blot was blocked overnight at 4° C. with 2% (w/v) BSA in phosphate buffer saline (PBS).

Probing was performed using pAb J108, which recognizes an epitope; FESFERGRECDAQCKKYDK, encoded by exon 3 within the amino-terminus of human lubricin/SZP and present in all alternatively spliced isoforms (Jay et al., J. Orthop. Res. 19:677-687, 2001). Incubation with pAb J 108 was conducted at 1:5,000 dilution in PBS+2% Tween-20 for 60 min at room temperature. Following washing with PBS+2% Tween-20, peroxidase-linked goat anti-rabbit immunoglobulin was added at a dilution of 1:10,000 for 60 min. Following exhaustive washing with PBS+2% Tween-20 and PBS, chemiluminescent substrate (Pierce, Rockford, Ill.) was added. Immunopositive bands were detected in a darkroom on BioMax film (Kodak, Rochester, N.Y.). Probing with peanut agglutinin (PNA) from Arachis hypogaea conjugated to peroxidase (Sigma-Aldrich) was performed at a concentration of 0.5 mg/mL in PBS+2% Tween-20 for 60 min at room temperature. Following exhaustive washing with PBS-2% Tween-20, and PBS, chemiluminescent substrate was added, and the blot was developed as described above.

### In Vitro Friction Assay of Human Lubricin and BSF

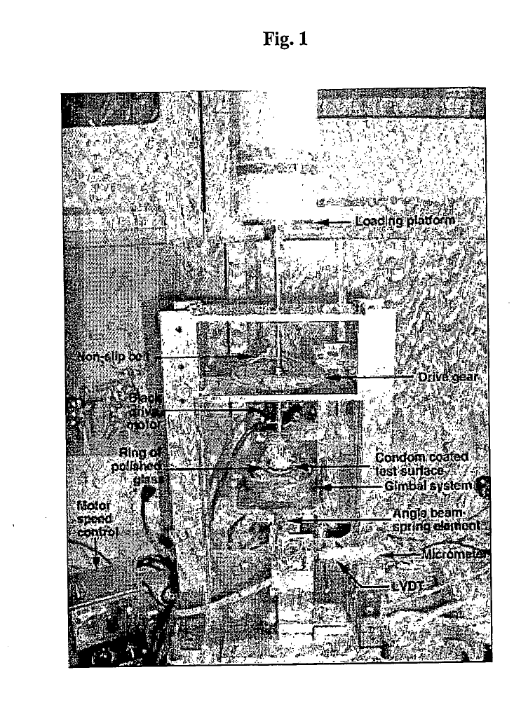

The boundary lubricating abilities of protease-treated human lubricin and BSF were measured using a friction apparatus as reported by Davis et al., J. Biomech. Eng. 101:185-192, 1979. The apparatus is shown in FIG. 1. Lubricant (200 μL) was applied between a bearing of latex and a ring of polished glass with a contact area of 1.59 cm². The latex was oscillated, under a pressure of 0.35×10⁶ N/m², against the polished glass with an entraining velocity of 0.37 mm/sec. The bearing system was axially loaded within a gimbals system free to rotate around two perpendicular horizontal axes. The friction apparatus recorded displacements of the gimbals system around the vertical loading axis through a linear displacement voltage transducer, where the output was directly proportional to the frictional torque (F). A bearing load of 70 newtons (N) was related to the coefficient of friction (μ) via Amonton's law, F=μN.

The μ of the lubricant was recorded at room temperature and was preceded by a baseline measurement of μ with normal saline (NS). Lubrication was manifested by a reduction in μ by the lubricant relative to that of NS. Negative Δμ (−Δμ) values indicate lubrication whereas positive Δμ (+Δμ) indicate friction. Addition of 200 μL of lubricant was followed by bringing the bearing surfaces close enough so that the solution wet both surfaces. After 5 min for equilibration, the latex-coated bearing was brought to rest on the glass as it was oscillating. Voltage measurements were recorded at 1, 3, and 5 min. After 5 min, the surfaces were separated for 2 min and then brought back together for three additional 5 min session. The 3 and 5 min μ values of the last two 5 min session were recorded and averaged, and data were later combined with another duplicate experiment providing 8 distinct measurements of μ.

### Pooling of OA, RA, and KJS SF Aspirates, Lubricin Supplementation, and Treatment with Protease Inhibitors

The SF aspirates from patients diagnosed with a Noyes criteria grade III or IV OA (n=60), with RA (n=20), or with KJS (n=23) were pooled in equal proportions. Pooled OA, RA, and KJS SF aspirates were supplemented with purified human lubricin until normal μ values were obtained. The lubricin-supplemented pooled OA, RA, and KJS SF aspirates were treated at 37° C. and sampled after 24, 48, and 96 hours. The friction analysis of the sampled SF was conducted as described above. Normal human SF was obtained from cartilage allograft donors and was utilized as a control.

Lubricin-supplemented pooled RA and KJS SF aspirates were treated with the following protease inhibitors: 1) E-64 to a final concentration of 10 μM; 2) Z-LLL-FMK (Sigma-Aldrich) to a final concentration of 20 μM; 3) PMSF (Sigma-Aldrich) to a final concentration of 25 μM; and 4) EDTA (Sigma-Aldrich) to a final concentration of 10 mM. The treatments were performed at 37° C., and SF was sampled at 24, 48, and 96 hours post-treatment. The in vitro friction assay of sampled SF was performed as described above.

### Ex Vivo μ of Excised Murine Joints Following Protease Treatment

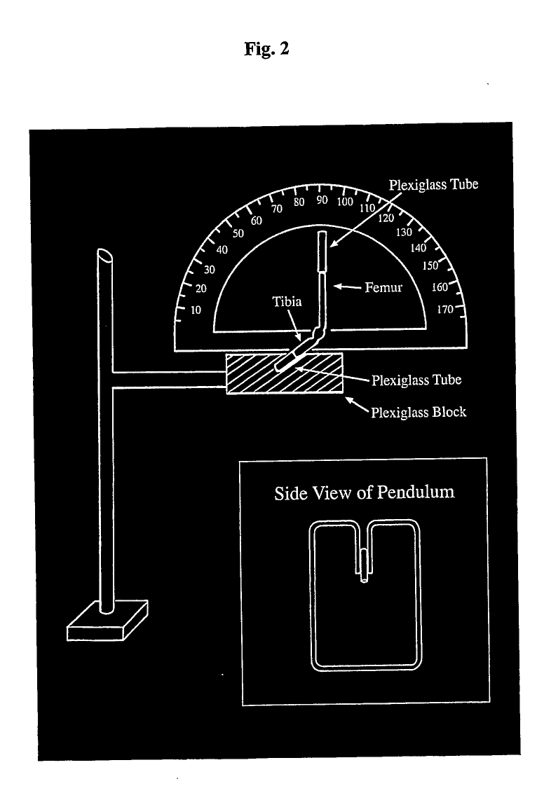

Murine joint friction measurements were performed ex vivo in a modified Stanton pendulum configuration (Chamely, New Scientist 6:60, 1959) illustrated in FIG. 2. Excised murine joints, from 8 week old Svev129 mice, weighing approximately 20 grams (Taconic, Germantown, N.Y.), were stripped of supporting connective tissue and musculature, while the synovium was left undisturbed. The femur and tibia were severed mid-length and covered with connecting plexiglass tubing. The center of the joint served as the axis of rotation of a 1 Hz pendulum. The joints were loaded with 20 grams and allowed to oscillate. The pendulum was set in motion at an angle of 30° off the perpendicular axis. The pendulum motion was videotaped and post-hoc analysis was performed to establish baseline μ. The deceleration of the pendulum, a=dv/dt, was used to calculate μ. Velocity (v) was calculated from the equation: v=(2 gh)^{1/2} where h is the height from where the pendulum reached apogee to the point of maximum velocity at a=0. The μ of murine joint was calculated to be equal a/g. Presently, this calculation neglects aerodynamic drag and assumes g equals 9.81 m/s². A 5 μL protease solution containing 0.05 U of CB, NE, α-chymotrypsin, or trypsin was delivered intra-articularly. The limbs were treated at room temperature for 2 hours and the limbs were subsequently allowed to oscillate to estimate μ following treatment with the protease.

### Measurement of CB Activity in OA, RA, and KJS SF Aspirates

Specific CB activity was assayed in a manner similar to the one reported previously (Huet et al., Clin. Chem. 38:1694-1697, 1992). Aliquots of OA, RA, and KJS SF (20 μL) were mixed with 40 μL of 300 μM fluorogenic substrate, Z-Arg-Arg-7-amino-4-methylcoumarin (AMC), in 0.25 M sodium acetate buffer, pH 5.5, containing 2 mM EDTA, 0.25 g/L Brij 35 and 1.25 mM dithiothreitol. The mixture was incubated for 10 min at 37° C. The reaction was stopped by adding 60 μL of a solution of 0.1 M iodoacetic acid and sodium acetate. Release of AMC was measured by a fluorocounter (Packard Instruments, Meriden, Conn.), using 360 nm and 485 nm as the excitation and emission wavelengths, respectively.

In a separate set of assays, E-64, a CB inhibitor (10 μM, Sigma-Aldrich), was included in the assay buffer to quantify CB activity. A standard curve was constructed from serial dilutions of AMC and the activity was expressed in units, where 1 unit corresponds to the release of 1 μmole of AMC per min.

### Statistical Analysis

Changes in μ of purified human lubricin and whole BSF following protease treatment are reported as average±standard deviation. CB activities in SF from OA, RA, and KJS SF are represented by box plots. The solid line represents the median, the box represents the middle 50% of the values, the error bars represent the 10^{th} and 90^{th} percentile and individual points represent outlying values. Significance testing was conducted by Student's t-test. The significance level was determined a priori to be α=0.05.

### Results

### Effects of CB, NE, α-Chymotrypsin, and Trypsin on Purified Human Lubricin

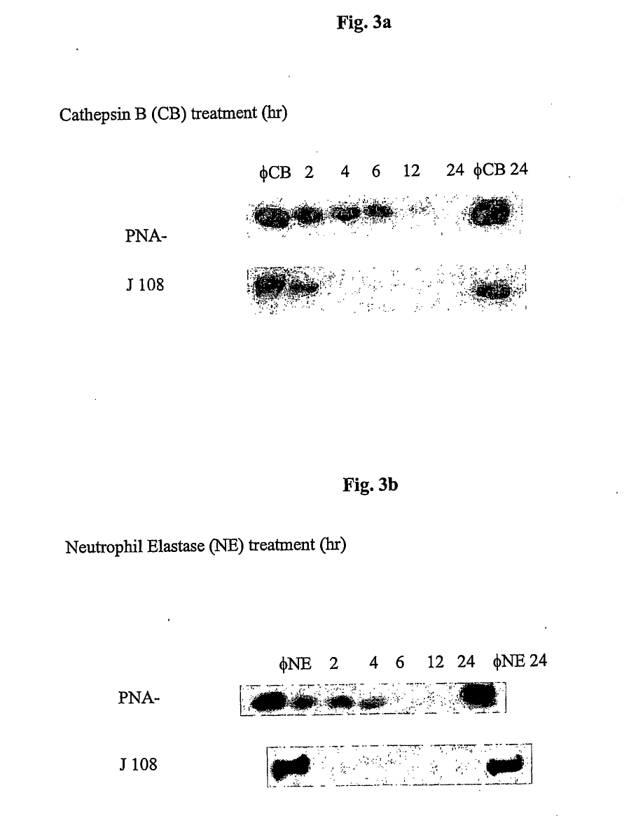

Treatment with 0.5 U/mL CB or NE resulted in a time-dependent degradation of lubricin, as illustrated by diminishing band intensities at 2, 4, 6, 12, and 24 hours post-treatment (FIGS. 3a and 3b). PNA positive bands were still detectable up to 6 hours after treatment of CB with human lubricin. However, at 12 and 24 hours, PNA reactivity was lost, indicating that β(1-3)Gal-GalNAc O-linked to the central exon 6 was completely degraded. On the contrary, no pAB J108 immunoreactive bands were detectable following treatment with CB for four hours, even with prolonged exposure (˜30 min) (FIG. 3a). Similar results were obtained with NE (FIG. 3b). In NE-treated human lubricin, no pAb J108 immunoreactive bands were detected following treatment for 2 hours at 37° C. Treatment with α-chymotrypsin or trypsin resulted in complete loss of PNA and J108 reactivity after 2 hours of treatment (data not shown). In all treatment experiments with human lubricin, no low molecular weight lubricin degradation products were detected using either PNA or pAb J108.

### Effects of CB, NE, α-Chymotrypsin, and Trypsin on In Vitro Lubricating Ability (Δμ of Human Lubricin and BSF

The Δμ values of human lubricin and BSF following time-dependent treatment using 0.5 U/mL of CB, NE, α-chymotrypsin, or trypsin are reported in Tables 1 and 2. The boundary lubricating ability of human lubricin progressively deteriorated following CB or NE treatment, as evidenced by a +Δμ following 12 and 24 hours treatment. A +Δμ was observed following 2 hour treatment with 0.5 U/mL of either α-chymotrypsin or trypsin, and continued to indicate friction for the duration of the treatment (Table 1). By contrast, the untreated control of human lubricin continued to exhibit a consistently −Δμ value over the same 24 hour period.

Treatment of human lubricin with CB resulted in a significant increase in Δμ at 4, 6, 12, and 24 hours, compared to undigested human lubricin control (P<0.001). Treatment of human lubricin with NE, α-chymotrypsin, or trypsin also resulted in a significant increase in Δμ at 2, 4, 6, 12, and 24 hours, compared to control (P<0.001).

The lubricating ability of BSF progressively diminished, as evidenced by a +Δμ following treatment with CB for 12 and 24 hours; following treatment with NE for 4, 6, 12, and 24 hours; following treatment with α-chymotrypsin for 6, 12, and 24 hours; or following treatment with trypsin for 6, 12, and 24 hours (Table 2). Treatment of BSF with CB, NE, α-chymotrypsin, or trypsin resulted in a significant (P<0.001) increase in Δμ at 2, 4, 6, 12, and 24 hours, compared to a BSF control.

TABLE 1
Changes in coefficient of friction (Δμ ± S.D.) of purified human lubricin following
treatment with 0.5 U/mL of cathepsin B, neutrophil elastase, α-chymotrypsin, or trypsin.

| | | Cathepsin B | Neutrophil Elastase | α-Chymotrypsin | Trypsin |
|---|---|---|---|---|---|
| | | (0.5 U/mLl) | (0.5 U/mL) | (0.5 U/mL) | (0.5 U/mL) |
| Lubricin+ | Δμ ± S.D.* | Δμ ± S.D.* | Δμ ± S.D.* | Δμ ± S.D.* | Δμ ± S.D.* |
| 0 hrs** | −0.068 ± 0.005 | −0.068 ± 0.005 | −0.068 ± 0.005 | −0.068 ± 0.005 | −0.068 ± 0.005 |
| 2 hrs | −0.061 ± 0.004 | −0.048 ± 0.006 | −0.038 ± 0.005 | 0.035 ± 0.006 | 0.025 ± 0.006 |
| 4 hrs | −0.059 ± 0.004 | −0.025 ± 0.005 | −0.019 ± 0.004 | 0.036 ± 0.003 | 0.032 ± 0.005 |
| 6 hrs | −0.059 ± 0.002 | −0.012 ± 0.003 | 0.004 ± 0.001 | 0.041 ± 0.003 | 0.038 ± 0.001 |
| 12 hrs | −0.061 ± 0.009 | 0.008 ± 0.001 | 0.015 ± 0.003 | 0.043 ± 0.003 | 0.046 ± 0.005 |
| 24 hrs | −0.060 ± 0.004 | 0.051 ± 0.004 | 0.054 ± 0.006 | 0.043 ± 0.003 | 0.049 ± 0.005 |

*Latex: glass bearing system Δμ values: an average of two experiments, each with 4 distinct Δμ measurements.
**Sampling was performed at 2, 4, 6, 12, and 24 hours following treatment with enzymes.

TABLE 2
Changes in coefficient of friction (Δμ ± S.D.) of bovine synovial fluid (BSF) following
treatment with 0.5 U/mL of cathepsin B, neutrophil elastase, α-chymotrypsin, or trypsin.

| | | Cathepsin B | Neutrophil Elastase | α-Chymotrypsin | Trypsin |
|---|---|---|---|---|---|
| | | (0.5 U/mL) | (0.5 U/mL) | (0.5 U/mL) | (0.5 U/mL) |
| BSF+ | Δμ ± S.D.* | Δμ ± S.D.* | Δμ ± S.D.* | Δμ ± S.D.* | Δμ ± S.D.* |
| 0 hrs** | −0.061 ± 0.005 | −0.061 ± 0.005 | −0.061 ± 0.005 | −0.061 ± 0.005 | −0.061 ± 0.005 |
| 2 hrs | −0.057 ± 0.004 | −0.046 ± 0.001 | −0.039 ± 0.001 | −0.033 ± 0.006 | −0.031 ± 0.005 |
| 4 hrs | −0.054 ± 0.002 | −0.029 ± 0.002 | 0.007 ± 0.001 | −0.012 ± 0.004 | −0.015 ± 0.002 |
| 6 hrs | −0.056 ± 0.002 | −0.017 ± 0.007 | 0.009 ± 0.001 | 0.006 ± 0.001 | 0.005 ± 0.001 |
| 12 hrs | −0.052 ± 0.003 | 0.004 ± 0.016 | 0.010 ± 0.001 | 0.010 ± 0.004 | 0.009 ± 0.003 |
| 24 hrs | −0.049 ± 0.001 | 0.048 ± 0.006 | 0.013 ± 0.002 | 0.013 ± 0.005 | 0.012 ± 0.004 |

*Latex: glass bearing system Δμ values: an average of two experiments, each with 4 distinct Δμ measurements.
**Sampling was performed at 2, 4, 6, 12, and 24 hours following treatment with enzymes.

### Effects of CB, NE, α-chymotrypsin, and Trypsin on Ex Vivo Lubricating Ability (μ) of Excised Murine Joints

The μ values of excised murine joints before and after intra-articular injection of 0.05 U each of CB, NE, α-chymotrypsin, and trypsin are illustrated in Table 3. Treatment with CB resulted in an average 73.7% increase in μ compared to an average 27.3% increase in μ following NE injection, an average 128.6% increase in μ following α-chymotrypsin injection, and an average 88.9% increase in μ following trypsin injection. There was a significant increase in μ of excised murine joints following CB injection (P<0.001), α-chymotrypsin injection (P<0.001), and trypsin injection (P<0.001), compared to the respective controls.

TABLE 3
Friction coefficients (μ) measurements of excised murine joints (n = 4) using
Stanton modified pendulum before and after intra-articular injection of 5 μl normal
saline and 5 μl containing 0.05 U each of cathepsin B (CB), neutrophil elastase
(NE), α-chymotrypsin, and trypsin followed by a 2 hour treatment at room temperature.

| | Saline | CB | NE | α-Chymotrypsin | Trypsin |
|---|---|---|---|---|---|
| | μ ± S.D. | μ ± S.D. | μ ± S.D. | μ ± S.D. | μ ± S.D. |
| Before | 0.0017 ± 0.0004 | 0.0019 ± 0.0005 | 0.0022 ± 0.0005 | 0.0021 ± 0.0005 | 0.0018 ± 0.0001 |
| After | 0.0017 ± 0.0002 | 0.0033 ± 0.0005 | 0.0028 ± 0.0008 | 0.0048 ± 0.0003 | 0.0034 ± 0.0003 |

### Effect of Protease Inhibitors on In Vitro Lubricating Ability (Δμ) of Pooled KJS and RA SF Aspirates Following Lubricin Supplementation

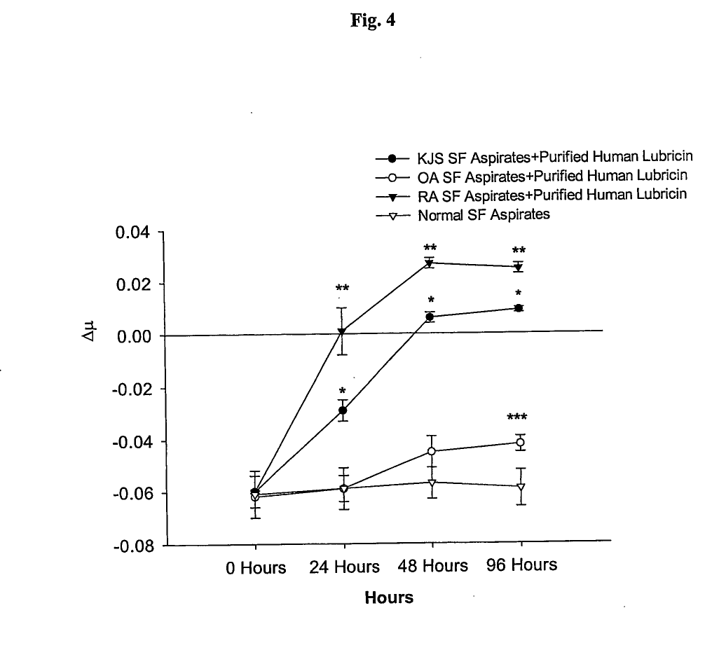

Normal lubrication of SF in pooled KJS, RA, and OA aspirates was established by the addition of lubricin. The time-dependent changes in lubricating ability of lubricin-supplemented KJS, RA, and OA SF following treatment at 37° C. for 24, 48, and 96 hours are illustrated in FIG. 4. The lubricin-supplemented pooled KJS and RA SF aspirates progressively lost their lubricating ability as demonstrated by +Δμ values following treatment for 48 and 96 hours. By contrast, the lubricin-supplemented OA SF aspirates continued to exhibit −Δμ values following treatment for 24, 48, and 96 hours, indicating lubrication.

The lubricin-supplemented KJS and RA SF aspirates exhibited a significantly higher Δμ value following 24, 48, and 96 hours treatment at 37° C., compared to normal SF treated for equivalent time intervals (P<0.001, FIG. 4). The lubricin-supplemented OA SF aspirates exhibited a significantly higher Δμ value following 96 hours of treatment at 37° C., compared to normal SF treated for equivalent time intervals (P<0.001, FIG. 4).

Changes in the lubricating ability of lubricin-supplemented pooled KJS SF aspirates in the presence of E-64, Z-LLL-FMK, PMSF, or EDTA are shown in Table 4. Treatment with E-64 resulted in an average 32.7% increase in Δμ at 24 hours, compared to an average 38.5% increase at 48 hours and an average 42.4% increase at 96 hours. Treatment with Z-LLL-FMK resulted in an average 41.7% increase in Δμ at 24 hours, compared to an average 48.3% increase at 48 hours and an average 53.3% increase at 96 hours. Treatment with PMSF resulted in an average 45.0% increase in Δμ at 24 hours, compared to an average 50.0% increase at 48 hours and an average 55.0% increase at 96 hours. Treatment with EDTA resulted in an average 50.0% increase at 24 hours, compared to an average 80.0% increase at 48 hours and an average 85.0% increase at 96 hours.

Treatment with E-64, Z-LLL-FMK, or PMSF preserved lubricating ability in lubricin-supplemented pooled KJS SF aspirates at 24, 48, and 96 hours. In each case, −Δμ was significantly (P<0.001) lower compared to controls without enzyme inhibitors. Treatment with 10 mM EDTA also resulted in a significant (P<0.01) decrease in Δμ compared to lubricin-supplemented pooled KJS SF aspirates at 48, and 96 hours. However, the preservation of lubricating ability was not as marked as observed in the treatments of aspirates with enzyme inhibitors.

The changes observed in lubricating ability of lubricin-supplemented pooled RA SF aspirates in the presence of E-64, Z-LLL-FMK, PMSF, and EDTA are provided in Table 5. Treatment with E-64 resulted in an average 11.7% increase in Δμ at 24 hours, compared to an average 60.0% increase at 48 hours and an average 76.7% increase at 96 hours. Treatment with Z-LLL-FMK resulted in an average 18.3% increase in Δμ at 24 hours, compared to an average 68.3% increase at 48 hours and an average 91.7% increase at 96 hours. Treatment with PMSF resulted in an average 85.0% increase in Δμ at 24 hours, compared to an average 86.3% increase at 48 hours and an average 85.0% increase at 96 hours. Treatment with EDTA resulted in an average 90.0% increase at 24 hours, compared to an average 88.3% increase at 48 hours and an average 128.3% increase at 96 hours.

Treatment with E-64, Z-LLL-FMK, or PMSF resulted in a significant decrease in Δμ, compared to lubricin-supplemented pooled RA SF aspirates at 24, 48, and 96 hours (P<0.001). Treatment with EDTA resulted in a significant decrease in Δμ, compared to lubricin-supplemented pooled RA SF aspirates at 48, and 96 hours (P<0.01).

TABLE 4
Changes in coefficient of friction (Δμ ± S.D.) of pooled
knee joint synovitis (KJS) synovial fluid (SF) aspirates supplemented
with purified human lubricin in the presence of protease inhibitors.

| | | Pooled KJS SF | Pooled KJS SF | Pooled KJS SF | Pooled KJS SF |
|---|---|---|---|---|---|
| | Pooled KJS SF | (n = 23) + | (n = 23) + | (n = 23) + | (n = 23) + |
| | (n = 23) + | Purified | Purified | Purified | Purified |
| | Purified | Lubricin + | Lubricin + | Lubricin + | Lubricin + |
| | Lubricin | E-64 | Z-LLL-FMK | PMSF | EDTA |
| | (*Δμ ± S.D.) | (*Δμ ± S.D.) | (*Δμ ± S.D.) | (*Δμ ± S.D.) | (*Δμ ± S.D.) |
| 0 hrs** | −0.060 ± 0.006 | −0.059 ± 0.004 | −0.060 ± 0.002 | −0.060 ± 0.001 | −0.060 ± 0.003 |
| 24 hrs | −0.029 ± 0.004 | −0.042 ± 0.005 | −0.035 ± 0.002 | −0.033 ± 0.006 | −0.030 ± 0.001 |
| 48 hrs | 0.006 ± 0.002 | −0.039 ± 0.007 | −0.031 ± 0.003 | −0.030 ± 0.005 | −0.012 ± 0.003 |
| 96 hrs | 0.009 ± 0.001 | −0.034 ± 0.002 | −0.028 ± 0.002 | −0.027 ± 0.005 | −0.009 ± 0.001 |

*Latex: glass bearing Δμ values: an average of two experiments, each with 4 distinct Δμ measurements.
**Sampling was performed at 24, 48, and 96 hours after treatment with inhibitors.

TABLE 5
Changes in coefficient of friction (Δμ ± S.D.) of pooled
rheumatoid arthritis (RA) synovial fluid (SF) aspirates supplemented
with purified human lubricin in the presence of protease inhibitors.

| | | Pooled RA SF | Pooled RA SF | Pooled RA SF | Pooled RA SF |
|---|---|---|---|---|---|
| | Pooled RA SF | (n = 45) + | (n = 45) + | (n = 45) + | (n = 45) + |
| | (n = 45) + | Purified | Purified | Purified | Purified |
| | Purified | Lubricin + | Lubricin + | Lubricin + | Lubricin + |
| | Lubricin | E-64 | Z-LLL-FMK | PMSF | EDTA |
| | (*Δμ ± S.D.) | (*Δμ ± S.D.) | (*Δμ ± S.D.) | (*Δμ ± S.D.) | (*Δμ ± S.D.). |
| 0 hrs** | −0.060 ± 0.002 | −0.060 ± 0.002 | −0.060 ± 0.002 | −0.060 ± 0.002 | −0.060 ± 0.002 |
| 24 hrs | 0.001 ± 0.009 | −0.053 ± 0.004 | −0.049 ± 0.002 | −0.009 ± 0.009 | −0.006 ± 0.004 |
| 48 hrs | 0.027 ± 0.002 | −0.024 ± 0.002 | −0.019 ± 0.001 | −0.008 ± 0.004 | 0.007 ± 0.004 |
| 96 hrs | 0.025 ± 0.002 | −0.014 ± 0.004 | −0.005 ± 0.003 | −0.009 ± 0.001 | 0.017 ± 0.001 |

*Latex: glass bearing Δμ values: an average of two experiments, each with 4 distinct Δμ measurements.
**Sampling was performed at 24, 48, and 96 hours after treatment with inhibitors.

### CB Activity in KJS, OA, and RA SF Aspirates

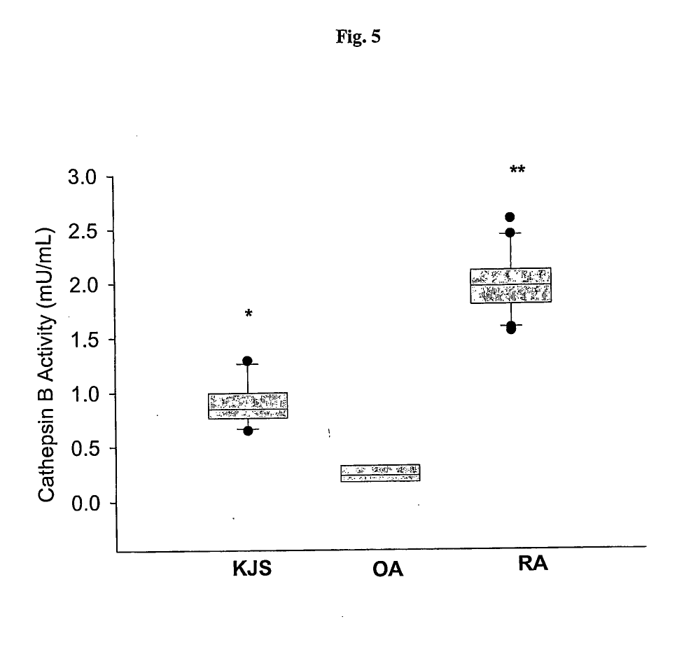

The CB activities in the SF aspirates from patients with KJS, OA, and RA are illustrated in FIG. 5. The CB activity in KJS SF aspirates was significantly higher than that observed in OA SF aspirates (P<0.005). On the other hand, the CB activity in RA SF aspirates was significantly higher than that observed in KJS aspirates (P<0.005) or OA aspirates (P<0.001).

### Discussion

CB or NE can proteolytically degrade lubricin in a time-dependent manner, as shown electrophoretically in the protease treatment of human lubricin and BSF, by a diminishing ˜240 KDa lubricin band intensity when probed with pAb J108 and PNA. The loss of the N-terminal exon 3 likely precedes damage to the central exon 6 as indicated by complete loss of pAb J108 epitope following 4 hours of CB and 2 hours following NE treatment. By contrast, the central exon 6 was still detectable following 6 hours of treatment with either enzyme. The boundary lubricating ability of purified lubricin treated with CB or NE continued to decline until it was completely lost by 12 hours. After 6 hours of CB or NE treatment, the ability of lubricin to lubricate was still evident by a −Δsupplemented value, albeit to a diminished extent. It therefore appears that boundary lubricating ability of lubricin is not dependent on the N-terminus. Loss of boundary lubricating ability of lubricin is more likely associated with loss of central exon 6 integrity and/or the C-terminus following CB and NE treatments. This is in agreement with previous reports that linked lubricating ability to β(1-3)Gal-GalNAc O-linked to threonine residues in the central exon 6 (Jay et al., Gluconj. J. 18:807-815, 2001).

Results of CB or NE treatment of BSF were similar to these of purified human lubricin. The proteolytic activity of these enzymes appeared to be restrained in BSF as CB or NE-treated BSF continued to lubricate at 12 hours post-treatment. This can be explained by 1) the presence of a myriad of other proteins that can serve as substrates to these enzymes, such as, for example, fibronectin; 2) the viscous nature of BSF, which may hinder proper interaction between the enzyme and substrate; and 3) the presence of endogenous proteolytic inhibitors. The increase in whole joint μ following intra-articular delivery of CB offers corroborating evidence of the ability of CB to digest lubricin in not only aspirated SF, but also in excised joints under load. These supporting experiments are important as they demonstrate the ability of CB to increase friction between loaded articular surfaces in their natural environment. Loss of SF's boundary lubricating ability has been considered thus far a surrogate indicator of the frictional properties within opposed and pressurized articular surfaces of the diarthrodial joint. The effects of CB on whole joint friction point to the prominent role that lubricin plays in maintaining low whole joint μ values.

Supplementation of KJS and RA SF aspirates with purified human lubricin did not re-establish normal lubricating ability over a 48 hour period of treatment at 37° C. By contrast, lubricating ability of similarly treated OA SF aspirates did not significantly change over 48 hours. This result indicates that lubricin is being degraded by proteases in KJS and RA SF aspirates that are in significant abundance compared to their levels in OA SF aspirates.

There is a significant contribution from cysteine proteases to the proteolytic destruction of lubricin by pooled KJS and RA SF aspirates. The compound E-64 (Barrett et al., Biochem. J 201:189-198, 1982), a broad spectrum irreversible inhibitor of cysteine proteases, prevented loss of boundary lubrication of lubricin-supplemented KJS and RA SF aspirates over 96 hours. In the present invention, E-64 was used at a concentration that would inhibit cysteine proteases, but would not be expected to inhibit either NE or metalloproteinases. Although the compound Cbz-Leu-Leu-Leu-fluoromethylketone (Z-LLL-FMK) possesses both CB and cathepsin L inhibitory activity, the similar effect of Z-LLL-FMK on the boundary lubricating ability of lubricin-supplemented KJS and RA SF aspirates is probably due to CB and not cathepsin L. Although cathepsin B and L expression is increased in the synovium of patients in early RA (Cunnane et al., Rheumatology 38:34-42, 1999), CB is detected in higher levels in SF from RA patients compared to cathepsin L (Ikeda et al., J. Med. Invest. 47:61-75, 2000). Furthermore, cathepsin L requires low pH to be activated, contrary to CB, which is proteolytically active near neutral pH. Compared to EDTA, NE inhibition by PMSF retarded the loss of boundary lubricating ability of lubricin-supplemented KJS and RA SF aspirates. This indicates that NE plays a more significant role in the proteolytic degradation of lubricin in these disease states compared to metalloproteinases. The results provided herein demonstrate that inhibition of CB or NE activity may prevent the proteolytic degradation of lubricin and the resultant loss of the SF's chondroprotective properties.

All publications and patents cited in this specification are hereby incorporated by reference herein as if each individual publication or patent were specifically and individually indicated to be incorporated by reference. Although the foregoing invention has been described in some detail by way of illustration and example for purposes of clarity of understanding, it will be readily apparent to those of ordinary skill in the art in light of the teachings of this invention that certain changes and modifications may be made thereto without departing from the spirit or scope of the appended claims.

What is claimed is:

1. A method of lubricating a joint in a mammal, said method comprising contacting said joint with lubricin and administering to said mammal a compound that inhibits an enzyme selected from the group consisting of neutrophil elastase, cathepsin B, cathepsin K, cathepsin L, cathepsin S, papain, trypsin, chymotrypsin, subtilisin, pepsin, bromelain, ficin, Protease A, Protease B, Protease D, granzyme A, granzyme B, granzyme K, pepsin, thermolysin, pronase, dipeptidyl peptidase IV, and pancreatin.

2. The method of claim 1, wherein said joint is an articulating joint of a human.

3. The method of claim 1, wherein said enzyme is cathepsin B.

4. The method of claim 3, wherein said compound is an aldehyde, an alpha-ketocarbonyl compound, a halomethyl ketone, a diazomethyl ketone, an (acyloxy)methyl ketone, a ketomethylsulfonium salt, an epoxy succinyl compound, avinyl sulfone, an aminoketone, or a hydrazide.

5. The method of claim 3, wherein said inhibitor is E-64, Z-Leu-Leu-Leu-fluoromethyl ketone (Z-LLL-FMK), Z-Phe-Phe-fluoromethyl ketone, calpain inhibitor I, calpain inhibitor II, antipain, biotin-Phe-Ala-fluoromethyl ketone, cystatin, CA-074, CA-074 methyl ester, chymostatin, leupeptin, N-methoxysuccinyl-Phe-HOMO-Phe-fluoromethyl ketone, or a procathepsin B fragment.

6. The method of claim 1, wherein said enzyme is neutrophil elastase.

7. The method of claim 6, wherein said compound is PMSF, ICI 200,355, secretory leukoproteinase inhibitor, MeOSuc-Ala-Ala-Pro-Ala-CMK, Boc-Ala-Ala-Ala-NHO-Bz, or MeOSuc-Ala-Ala-Pro-Val-CMK.

8. The method of claim 1, wherein said compound is administered locally.

9. A method of inhibiting adhesion formation between a first surface and a second surface in a mammal, said method comprising placing lubricin between said first and second surfaces in an amount sufficient to prevent adhesion of said surfaces in said mammal and administering to said mammal a compound that inhibits an enzyme selected from the group consisting of neutrophil elastase, cathepsin B, cathepsin K, cathepsin L, cathepsin S, papain, trypsin, chymotrypsin, subtilisin, pepsin, bromelain, ficin, Protease A, Protease B, Protease D, granzyme A, granzyme B, granzyme K, pepsin, thermolysin, pronase, dipeptidyl peptidase IV, and pancreatin.

10. The method of claim 9, wherein said first surface and said second surface are both injured tissues of said mammal.

11. The method of claim 9, wherein said first or said second surface is an artificial device.

12. The method of claim 11, wherein said artificial device is an orthopedic implant.

13. The method of claim 9, wherein said lubricin is present in a composition, said composition being in the form of a membrane, foam, gel, or fiber.

14. The method of claim 9, wherein said first and said second surfaces are tissues injured due to a surgical incision.

15. The method of claim 9, wherein said first and said second surfaces are tissues injured due to trauma.

16. The method of claim 9, wherein said enzyme is cathepsin B.

17. The method of claim 16, wherein said compound is an aldehyde, an alpha-ketocarbonyl compound, a halomethyl ketone, a diazomethyl ketone, an (acyloxy)methyl ketone, a ketomethylsulfonium salt, an epoxy succinyl compound, avinyl sulfone, an aminoketone, or a hydrazide.

18. The method of claim 16, wherein said inhibitor is E-64, Z-Leu-Leu-Leu-fluoromethyl ketone (Z-LLL-FMK), Z-Phe-Phe-fluoromethyl ketone, calpain inhibitor I, calpain inhibitor II, antipain, biotin-Phe-Ala-fluoromethyl ketone, cystatin, CA-074, CA-074 methyl ester, chymostatin, leupeptin, N-methoxysuccinyl-Phe-HOMO-Phe-fluoromethyl ketone, or a procathepsin B fragment.

19. The method of claim 9, wherein said enzyme is neutrophil elastase.

20. The method of claim 19, wherein said compound is PMSF, ICI 200,355, secretory leukoproteinase inhibitor, MeOSuc-Ala-Ala-Pro-Ala-CMK, Boc-Ala-Ala-Ala-NHO-Bz, or MeOSuc-Ala-Ala-Pro-Val-CMK.

21. A pharmaceutical composition comprising lubricin; a compound that inhibits an enzyme selected from the group consisting of neutrophil elastase, cathepsin B, cathepsin K, cathepsin L, cathepsin S, papain, trypsin, chymotrypsin, subtilisin, pepsin, bromelain, ficin, Protease A, Protease B, Protease D, granzyme A, granzyme B, granzyme K, pepsin, thermolysin, pronase, dipeptidyl peptidase IV, and pancreatin; and a pharmaceutically acceptable excipient.

22. The pharmaceutical composition of claim 21, wherein said enzyme is cathepsin B.

23. The pharmaceutical composition of claim 21, wherein said enzyme is neutrophil elastase.
